(19) United States
(12) Patent Application Publication
(10) Pub. No.: US 2008/0182789 A1
(43) Pub. Date: Jul. 31, 2008

(54) METHOD OF TREATING PULMONARY FIBROSIS

(75) Inventor: John McMichael, Delanson, NY (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Milkhaus Laboratory, Inc., Delanson, NY (US)

(21) Appl. No.: 11/966,487

(22) Filed: Dec. 28, 2007

Related U.S. Application Data
(60) Provisional application No. 60/898,285, filed on Jan. 30, 2007.

(51) Int. Cl. A61K 38/16; A61P 11/00

(52) U.S. Cl. 514/12

(57) ABSTRACT

Methods and compositions for treating pulmonary fibrosis are presented. Methods comprise administering compositions comprising DNA and streptolysin O to a subject in a manner so as not to effect gene transfer.

### RELATED APPLICATION DATA

This application claims the benefit of U.S. Provisional Application No. 60/898,285, entitled “Methods for Treatment of Pulmonary Fibrosis,” filed on Jan. 30, 2007, which is hereby incorporated by reference herein in its entirety.

### FIELD OF THE INVENTION

The present invention relates generally to improved methods for treating pulmonary fibrosis.

### BACKGROUND OF THE INVENTION

Pulmonary fibrosis is a common consequence and often a central feature of many lung diseases. In some disorders fibrosis develops focally and to a limited degree. For example, in asthma and chronic obstructive pulmonary disease fibrotic changes occur around conducting airways where scarring may be important to the pathophysiology.

The diagnosis of these conditions can usually be made by careful history, physical examination, chest radiography, including a high resolution computer tomographic scan (HRCT), and open lung or transbronchial biopsies. However, in a significant number of patients, no underlying cause for the pulmonary fibrosis can be found. These conditions of unknown etiology have been termed idiopathic interstitial pneumonias. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

Idiopathic pulmonary fibrosis (IPF) is clinically a restrictive lung disease that characteristically progresses relentlessly to death from respiratory failure. Median survival of newly diagnosed patients with IPF is about 3 years, similar to that of clinical stage 1b non-small cell lung cancer. The quality of life for IPF patients is also poor. Despite this, there has been remarkably little progress in development and/or assessment of therapeutic strategies for IPF.

Of interest to the present invention are the disclosure of co-owned U.S. Pat. Nos. 5,726,160, 5,948,768 and 5,955,442, the disclosures of which are incorporated by reference herein, which are directed to method of treatment of respiratory congestion, other respiratory distress and otitis media by administration of DNA in a pharmaceutically-acceptable vehicle in a manner so as not to effect gene transfer.

Also of interest to the present invention is the disclosure of co-owned U.S. Pat. No. 5,736,508 the disclosure of which is hereby incorporated by reference which relates to the use of streptolysin O in methods for treatment of scar tissue such as caused by surgery, acne, burns and trauma. Streptolysin O is one of a group of filterable hemolysins derived from Group A beta-hemolytic streptococci. Specifically, streptolysin O is a 60 kD peptide which is hemolytic in its reduced state but is inactivated upon oxidation. Streptolysin O is generally used in the art as an analytical reagent for permeabilizing cells. See, e.g., Razin et al., Proc. Nat'l. Acad. Sci. (USA), 91:7722-7726 (1994).

### SUMMARY OF THE INVENTION

The present invention is based on the discovery that the combination of streptolysin O with DNA is unusually effective in alleviating the symptoms of pulmonary fibrosis.

In one aspect, the invention provides a method of treating pulmonary fibrosis in a subject comprising administering to said subject an effective amount of a composition comprising DNA and streptolysin O, wherein the DNA is administered in a manner so as not to effect gene transfer.

While the precise dosage of the DNA and streptolysin O components of the composition of the invention will vary from individual to individual, they may be readily determined empirically. Nevertheless, preferred dosages of streptolysin O according to the invention generally range from about 0.0032 units to about 50 units with dosages of from 0.01 units to 10 units being preferred. Preferred dosages of DNA range from about 0.1 μg to about 0.003 mg DNA per dose with dosages of about 0.3 μg of DNA per dose being particularly preferred.

The compositions of the invention may be administered by a variety of routes including intravenous, intramuscular, subcutaneous, intrathecal, and oral routes of administration with sublingual administration being preferred. It is also anticipated that alternative routes of administration for treatment of respiratory diseases may be by inhalation. If administered sublingually, it is preferred that the compositions be administered 1-10 drops (0.05 ml per drop) per day with a dosage of from about 0.01 to about 10 units of streptolysin O and from about 0.1 μg to about 0.003 mg DNA per drop being preferred.

Suitable pharmaceutically acceptable vehicles for the active components of the invention are well known to those of skill in the art but preferably include those selected from the group consisting of water, saline, albumin and dextrose.

### DETAILED DESCRIPTION OF THE INVENTION

The present invention provides methods for treating respiratory illness. Specifically, the invention provides methods for treating pulmonary fibrosis by administering a composition comprising streptolysin O and DNA in an amount effective to treat pulmonary fibrosis.

A preferred route of administration is sublingual, but other routes, such as subcutaneous, intravenous, intramuscular, and intrathecal are expected to work. DNA for use in the present invention may be prokaryotic DNA or eukaryotic DNA such as salmon testicle DNA or calf thymus DNA (Sigma, St. Louis) and may be formulated in a number of pharmaceutically-acceptable vehicles, including water, saline, albumin, and dextrose.

Pulmonary function tests may be employed to detect physiological changes associated with the presence of pulmonary disease. Pulmonary function tests performed in a clinical setting may be used to evaluate lung mechanics, gas exchange, pulmonary blood flow, and blood gases and pH. They are used to evaluate patients in the diagnosis of pulmonary disease, assessment of disease development, or evaluation of the risk of pulmonary complications from surgery.

The term “pulmonary function tests” is used to indicate a battery of studies or maneuvers that may be performed using standardized equipment to measure lung function. Pulmonary function tests include simple screening spirometry, formal lung volume measurement, diffusing capacity for carbon monoxide, and arterial blood gases.

The pulmonary function tests may obtain such values as FEV (forced expiratory volume), FVC (forced vital capacity), FEF_{25%-75%} (forced expiratory flow rate), PEFR (peak expiratory flow rate), FRC (functional residual capacity), RV (residual volume), TLC (total lung capacity), and/or flow/volume loops. FEV measures the volume of air exhaled over a predetermined period of time by a forced expiration immediately after a full inspiration. FVC measures the total volume of air exhaled immediately after a full inspiration. FEF_{25%-75%} measures the rate of air flow during a forced expiration divided by the time in seconds for the middle half of expired volume. PEFR measures the maximum flow rate during a forced exhale starting from full inspiration. FRC is the volume of air remaining in the lungs after a full expiration. RV is the FRC minus the expiratory reserve volume. TLC is the total volume in the lungs at the end of a full inspiration. Flow/volume loops are graphical presentations of the percent of total volume expired (on the independent axis) versus the flow rate during a forced expiratory maneuver. Normal values and lower limits of normal can be determined as defined by Hankinson et al (the National Health and Nutrition Examination Survey [NHANES] III predicted set).

The following Example illustrates the preferred embodiments of the invention and provides evidence of the effectiveness of claimed treatment methods. According to this example, human subjects diagnosed with pulmonary fibrosis were treated with compositions comprising DNA and streptolysin O according to the invention Numerous improvements and further aspects of the invention are apparent to the skilled artisan upon consideration of the Example which follows.

### EXAMPLE

According to this example, a 72-year old male, diagnosed with idiopathic pulmonary fibrosis and showing marked progression of disease as evidenced by compromised pulmonary function tests, ability to carry out the normal routines of daily life, and cardiac changes associated with compromised lungs, began therapy with the composition of the invention. Specifically, the composition comprising 2 units of streptolysin O and 0.3 μg of Salmon testicle DNA (Sigma/Aldrich) was administered sublingually four times daily in dosages of one drop (0.05 mL).

Every six months the patient was subjected to a variety of tests including spirometry, lung volume and diffusion capacity tests in order to evaluate the progression of the disease. Each test provided results within the normal limits. Thus, the subject observed stable or improved pulmonary function tests and an improved quality of life and did not exhibit any signs of disease progression.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. A method for treating one or more symptoms of pulmonary fibrosis in a subject suffering from pulmonary fibrosis, comprising the step of:
administering to said subject in amounts effective to treat one or more symptoms of pulmonary fibrosis a composition comprising DNA and streptolysin O, wherein the composition is administered in a manner so as not to effect gene transfer.

2. The method according to claim 1, wherein said composition is administered sublingually in the form of a liquid drop.

3. The method according to claim 1, wherein the composition comprises from about 0.1 μg to about 0.003 mg DNA per dose.

4. The method according to claim 1, wherein said composition comprises about 0.3 μg of DNA per dose.

5. The method according to claim 1, wherein said composition comprises from about 0.0032 units to about 50 units of streptolysin O per dose.

6. The method according to claim 1, wherein said composition comprises from about 0.01 units to about 10 units of streptolysin O per dose.

7. The method according to claim 1 wherein said composition is administered by a route selected from the group consisting of sublingual, subcutaneous, intravenous, intramuscular, inhalation and intrathecal administration.

8. The method according to claim 8, wherein said composition is administered sublingually in the form of a liquid drop.

9. The method according to claim 1 wherein said patient is a human.

10. The method according to claim 1 wherein the pulmonary fibrosis is idiopathic.

11. The method according to claim 1, wherein the symptoms are selected from the group consisting of shortness of breath, chronic dry, hacking cough, fatigue and weakness, discomfort in the chest, loss of appetite and rapid weight loss.

12. A composition for treatment of one or more symptoms of pulmonary fibrosis comprising DNA and streptolysin O in amounts effective to treat one or more symptoms of pulmonary fibrosis and a pharmaceutically effective carrier.

13. The composition of claim 12, wherein said composition comprises from about from about 0.1 μg to about 0.003 mg DNA per dose.

14. The composition according to claim 12, wherein said composition comprises about 0.3 μg of DNA per dose.

15. The composition according to claim 12, wherein said composition comprises from about 0.0032 units to about 50 units of streptolysin O per dose.

16. The composition according to claim 12, wherein said composition comprises from about 0.01 units to about 10 units of streptolysin O per dose.
